(19) United States
(12) Patent Application Publication
(10) Pub. No.: US 2011/0022158 A1
(43) Pub. Date: Jan. 27, 2011

(54) Bioerodible Medical Implants

(75) Inventors: Liliana Atanasoska, Minneapolis, MN (US); Jonathan S. Stinson, Minneapolis, MN (US); Charles Deng, Chanhassen, MN (US); Dennis A. Boismier, Shorewood, MN (US); Jan Weber, Maastricht (NL); Torsten Scheuermann, Munich (DE)

Correspondence Address: FISH & RICHARDSON P.C. (BO), P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/507,171

(22) Filed: Jul. 22, 2009

(51) Int. Cl. A61F 2/06

(52) U.S. Cl. 623/1.38

(57) ABSTRACT

A medical implant includes a bioerodible portion adapted to degrade under physiological conditions. The bioerodible portion includes a bioerodible metal matrix and a salt or clay within the bioerodible metal matrix.

### TECHNICAL FIELD

This invention relates to bioerodible medical implants, and more particularly to bioerodible endoprostheses.

### BACKGROUND

A medical implant can replace, support, or act as a missing biological structure. Some examples of medical implants can include: orthopedic implants, bioscaffolding, endoprostheses such as stents, covered stents, and stent-grafts; bone screws, and aneurism coils. Some medical implants are designed to erode under physiological conditions.

Medical endoprostheses can, for example, be used in various passageways in a body, such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

A medical implant is described that includes a bioerodible portion adapted to degrade under physiological conditions. The bioerodible portion includes a bioerodible metal matrix and a salt or clay within the bioerodible metal matrix.

The salt can be a chloride salt, a fluoride salt, a sulfate, or a combination thereof In some embodiments, the salt has a melting point of greater than 700 degrees Celsius. For example, the salt can be iron chloride, magnesium chloride, sodium chloride, iron fluoride, sodium fluoride, sodium bicarbonate, sodium sulfate, calcium phosphate, magnesium acetate, magnesium citrate, potassium sulfate, lidocanine hydrochloride, dexamethasone sodium phosphate, paclitaxel mesylate, or a combination thereof.

The clay can be a calcium permanaganate (e.g., CaHMnₓO_{y}).

The bioerodible portion can be essentially free of polymer. In other embodiments, the bioerodible portion includes a polymer matrix within the bioerodible metal matrix with the salt or clay being within the polymer matrix. The polymer matrix can be a polymer selected from the group of poly(ethylene oxide), polylactic acid, poly(lactic-co-glycolic acid), polycaprolactone, polycaprolactone-polylactide copolymer, polycaprolactone-polyglycolide copolymer, polycaprolactone-polylactide-polyglycolide copolymer, polylactide, polycaprolactone-poly(β-hydroxybutyric acid) copolymer, poly(β-hydroxybutyric acid) and combinations thereof.

The bioerodible metal can be selected from the group of magnesium, iron, tungsten, zinc and alloys thereof In some embodiments, the bioerodible metal includes iron or an alloy thereof.

The bioerodible portion can have an erosion rate of greater than thirty micrometers per year when submerged in Ringer's solution at ambient temperature.

The medical implant can be a stent. In other embodiments, the medical implant can be bioscaffolding, an aneurysm coil, an orthopedic implant, or a bone screw. In some embodiments, the medical implant can consists essentially of the bioerodible portion.

In another aspect, a medical implant includes a bioerodible portion adapted to degrade under physiological conditions, where the bioerodible portion includes a bioerodible metal that degrades under physiological conditions and an agent that creates a localized acidic environment when exposed to a body fluid under physiological conditions. The localized acidic environment accelerates the erosion of the bioerodible metal in the vicinity of the localized acidic environment. The agent is selected from the group consisting of salts, clays, polymers, an combinations thereof.

In some embodiments, the agent is a salt. The salt can be iron chloride, magnesium chloride, sodium chloride, iron fluoride, sodium fluoride, sodium bicarbonate, sodium sulfate, calcium phosphate, magnesium acetate, magnesium citrate, lidocanine hydrochloride, dexamethasone sodium phosphate, paclitaxel mesylate, or a combination thereof. In other embodiments, the agent is a clay (e.g., calcium permanaganate). In other embodiments, the agent is a polymer having acidic functional groups selected from the group of carboxylic acid functional groups, a sulfuric acid functional groups, and combinations thereof. The polymer can be selected from the group of poly(ethylene oxide), polylactic acid, poly(lactic-co-glycolic acid), polycaprolactone, polycaprolactone-polylactide copolymer, polycaprolactone-polyglycolide copolymer, polycaprolactone-polylactide-polyglycolide copolymer, polylactide, polycaprolactone-poly(β-hydroxybutyric acid) copolymer, poly(β-hydroxybutyric acid) and combinations thereof.

The bioerodible metal can be selected from the group of magnesium, iron, tungsten, zinc and alloys thereof. In some embodiments, the bioerodible metal includes iron or an alloy thereof.

The agent can be within a matrix of the bioerodible metal. In other embodiments, the agent is deposited on an outer surface of the bioerodible metal. For example, the outer surface can include surface pits and the agent can be deposited within the surface pits.

The medical implant can be a stent. In other embodiments, the medical implant can be bioscaffolding, an aneurysm coil, an orthopedic implant, or a bone screw. In some embodiments, the medical implant can consists essentially of the bioerodible portion.

The details of one or more embodiments are set forth in the accompanying drawings and the description below. Other features, objects, and advantages will be apparent from the description and drawings, and from the claims.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of an embodiment of an expanded stent.

FIGS. 3A-3F depict cross-sectional views of different embodiments of a stent.

FIG. 4 depicts an example of a method of producing a stent.

FIG. 5A depicts a stent having corrosion enhancing regions on connectors between bands.

FIG. 5B depicts a stent after the erosion of the connectors between bands.

FIGS. 6A-6D depict how a stent strut erodes with and without spaced corrosion enhancing regions.

Like reference symbols in the various drawings indicate like elements.

### DETAILED DESCRIPTION

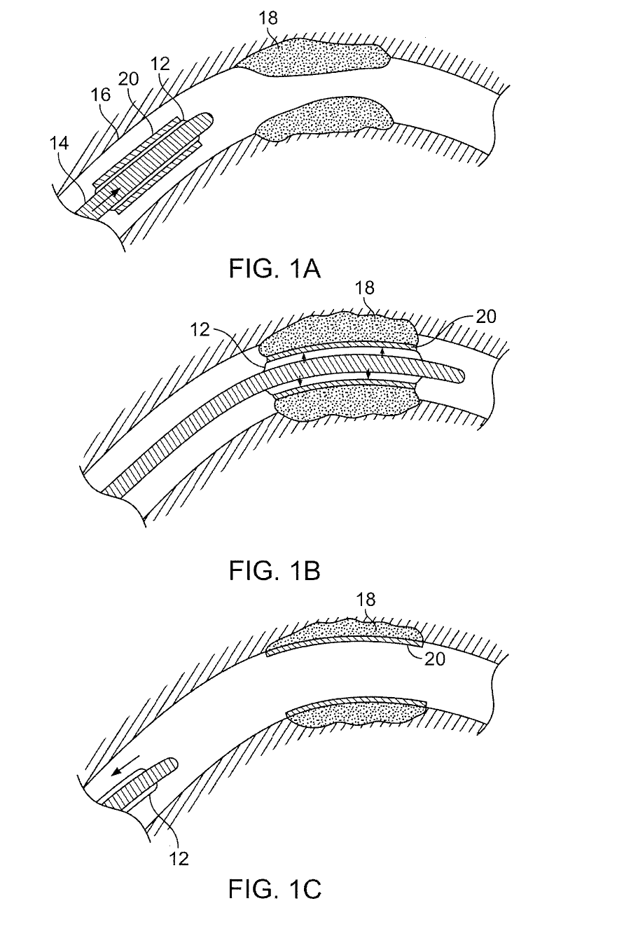

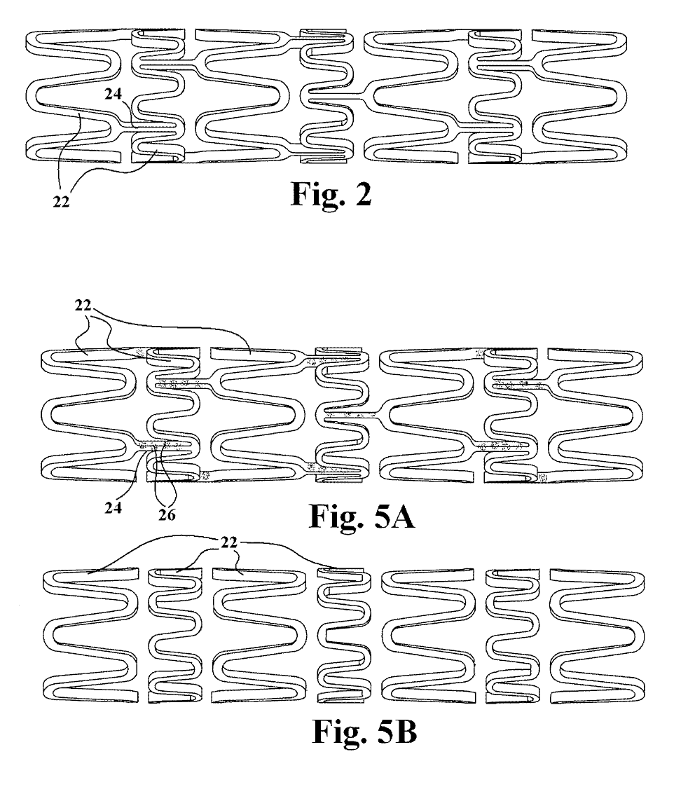

The medical implant can include one or more bioerodible portions adapted to degrade under physiological conditions. The bioerodible portions include a bioerodible metal and a salt, clay, and/or polymer to increase the erosion rate of the bioerodible metal. A stent 20, shown in FIGS. 1A-1C and 2, is discussed below as an example of one medical implant according to the instant disclosure. Other examples of medical implants can include orthopedic implants, bioscaffolding, bone screws, aneurism coils, and other endoprostheses such as covered stents and stent-grafts.

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C). In other embodiments, the stent 20 can be a self-expanding stent.

Referring to FIG. 2, a stent 20 can have a stent body having the form of a tubular member defined by a plurality of struts. The struts can include bands 22 and a plurality of connectors 24 that extend between and connect adjacent bands. During use, bands 22 can be expanded from an initial, smaller diameter to a larger diameter to contact stent 20 against a wall of a vessel, thereby maintaining the patency of the vessel. Connectors 24 can provide stent 20 with flexibility and conformability that allow the stent to adapt to the contours of the vessel. The stent 20 defines a flow passage therethrough and is capable of maintaining patency in a blood vessel.

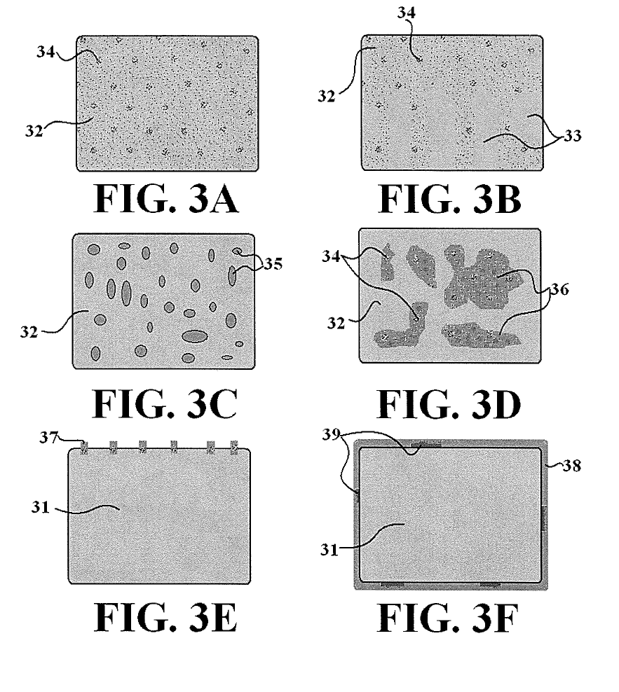

The stent 20 includes at least one bioerodible portion adapted to degrade under physiological conditions. The bioerodible portion includes a bioerodible metal and at least one of a salt, clay, or polymer increasing the erosion rate of at least a portion of the bioerodible portion. In some embodiments, the stent 20 can be entirely or almost entirely composed of the bioerodible portion. In other embodiments, a stent can include a bioerodible portion and other portions. In some embodiments, a bioerodible portion can include therapeutic agents that can be released as the bioerodible portion degrades. FIGS. 3A-3F depict examples of cross-sections of stent struts of a bioerodible portion of a stent according to different embodiments.

The bioerodible metal of the bioerodible portion erodes under physiological conditions. Examples of bioerodible metals include iron, magnesium, tungsten, zinc, and alloys thereof For example, the bioerodible metal can be a bioerodible iron alloy that includes up to twenty percent manganese, up to 10 percent silver, and up to five percent carbon. In other embodiments, the bioerodible metal includes iron alloyed with silicone (e.g., about three percent silicone). The bioerodible metal can also be a bioerodible magnesium alloy that includes up to nine percent aluminum, up to five percent rare earth metals, up to five percent zirconium, up to five percent lithium, up to five percent manganese, up to ten percent silver, up to five percent chromium, up to five percent silicon, up to five percent tin, up to six percent yttrium, and up to ten percent zinc. Suitable magnesium bioerodible alloys include ZK31, which includes three percent zinc and one percent zirconium; ZK61, which includes six percent zinc and one percent zirconium; AZ31, which includes three percent aluminum and one percent zinc; AZ91, which includes nine percent aluminum and one percent zinc; WE43, which includes four percent yttrium and three percent rare earth metals; and WE54, which includes five percent yttrium and four percent rare earth metals. In some embodiments, the stent 20 can include a body including one or more bioerodible metals, such as magnesium, zinc, iron, or alloys thereof.

The bioerodible portion can include a salt that ionizes to produce electrolytes when exposed to a body fluid within a physiological environment. For example, the salt can be a chloride salt, a fluoride salt, or a sulfate. Examples of chloride salts include iron chloride, magnesium chloride, potassium chloride, and combinations thereof. Examples of fluoride salts include iron fluoride, magnesium fluoride, potassium fluoride, and combinations thereof Dibasics and tribasics have only partially been neutralized (e.g., sodium bicarbonate, sodium sulfate, calcium phosphate) are also suitable for use as the salt. Other suitable salts include salts of Ca, Zn, Mn, Co as cations and phosphate, bicarbonates, manganates, and organic acids such as citrates, acetates, lactates, glycolates, and amino acids as anions. In some embodiments, magnesium acetate, magnesium citrate, or a combination thereof can be used as the salt.

The salt can be included within a matrix of the bioerodible metal material and/or deposited on a surface of the bioerodible metal. The ionization of the salt to produce electrolytes can accelerate the erosion rate of the bioerodible metal by increasing the conductivity of the body fluid surrounding the bioerodible metal. This increased conductivity can increase the efficiency of the oxidation/reduction reaction occurring on surfaces of the bioerodible metal exposed to the body fluid. Furthermore, some salts can ionize to alter the pH of the surrounding environment, which can also change the erosion rate to the bioerodible metal. The patterning of the salt within a bioerodible metal matrix and/or along the surface of the bioerodible metal can impact the overall erosion pattern of the bioerodible portion. In some embodiments, the salt is a salt form of a drug or therapeutic agent. For example, the salt can include protonated drugs bound to chloride ions (e.g., lidocanine hydrochloride). Other salt forms of therapeutic agents include dexamethasone sodium phosphate. In some embodiments, the salt includes a salt form of paclitaxel (e.g., paclitaxel mesylate).

The bioerodible portion can include a clay, such as a bioerodible clay that produces acidic byproducts when exposed to a body fluid within a physiological environment. In some embodiments, the clay is a calcium permanaganate (e.g., Hollandite or Rancieite). For example, CaHMnₓO_{y} erodes to produce an acidic environment when exposed to a body fluid within a physiological environment. Other suitable clays can be nitrates, borates, carbonates, or combinations thereof. For examples nitrocalcite (hydrated calcium nitrate), Nitro magnesite (hydrated calcium nitrate), admontite (hydrated magnesium borate), calciborate, aragonite (calcium carbonite), and barringtonite (hydrated magnesium carbonite) are suitable clays. The clay can be included within a matrix of the bioerodible metal material and/or deposited on a surface of the bioerodible metal. The patterning of the clay within a bioerodible metal matrix and/or along the surface of the bioerodible metal can impact the overall erosion pattern of the bioerodible portion. Clays that dissolve rapidly in water can also create an open porous metal framework helping the erosion process by producing a higher surface area.

The bioerodible portion can include a polymer. For example, polymers in the bioerodible portion can include poly-glutamic acid (“PGA”), poly(ethylene oxide) (“PEO”), polycaprolactam, poly(lactic-co-glycolic acid) (“PLGA”), polysaccharides, polycaprolactone (“PCL”), polycaprolactone-polylactide copolymer (e.g., polycaprolactone-polylactide random copolymer), polycaprolactone-polyglycolide copolymer (e.g., polycaprolactone-polyglycolide random copolymer), polycaprolactone-polylactide-polyglycolide copolymer (e.g., polycaprolactone-polylactide-polyglycolide random copolymer), polylactide, polycaprolactone-poly(β-hydroxybutyric acid) copolymer (e.g., polycaprolactone-poly(β-hydroxybutyric acid) random copolymer), poly(β-hydroxybutyric acid) or a combination thereof Additional examples of bioerodible polymers are described in U.S. Published Patent Application No. 2005/0251249, which is hereby incorporated by reference. In some embodiments, the polymer can include acidic functional groups that create a localized acidic environment when exposed to a body fluid. For example, some polymers can include carboxylic acid functional groups, sulfuric acid functional groups, phosphoric acid functional groups, nitric acid functional groups and combinations thereof. In some embodiments, the polymer can be loaded with a salt and/or a clay. Some polymers can swell when exposed to a body fluid and allow for fluid to contact acid producing components within the polymer, which can include acidic functional groups of the polymer, acid producing bioerodible clays, and acidic salts. In some embodiments, the polymer can form a galvanic couple with the bioerodible metal and act as a cathode to result in the preferential erosion of the bioerodible metal. A salt can be within a matrix of the polymer and can ionize when exposed to a body fluid to make the polymer conductive. The ionized salt within the polymer matrix can act as an electrolyte to increase the efficiency of the oxidation/reduction reaction of the galvanic couple between the bioerodible metal and the polymer to further accelerate the erosion of the bioerodible metal. The patterning of polymer, and any acidic functional groups within the polymer, can impact the overall erosion pattern of the bioerodible portion.

FIG. 3A depicts a first embodiment of a stent strut cross-section. The strut includes a bioerodible metal matrix 32 and a plurality of deposits 34 of a salt or clay. The deposits 34 can be embedded within the bioerodible metal matrix 32 inter-granularly, leading to a faster corrosion rate when the bioerodible metal portion is exposed to a body fluid. The presence of the deposits 34 can increase the erosion rate of the bioerodible metal by increasing the porosity of the bioerodible metal, by increasing the concentration of electrolytes in the body fluid, and/or by altering the pH of the body fluid surrounding different portions of the bioerodible metal. The increased porosity is an increase in surface area exposed to a body fluid, which allows for additional oxidation/reduction reaction sites. The increased concentration of electrolytes in the body fluid can make the body fluid more electrically conductive, which can increase the efficiency of any oxidation/reduction reactions taking place between different portions of the bioerodible metal. Additionally, some salts can alter the pH of the body fluid, which can also impact the erosion rate of the bioerodible metal. For example, a bioerodible metal matrix 32 having inter-granular salt deposits can have an in-vivo corrosion rate of greater than 30 micrometers per year. In some embodiments, the in-vivo corrosion rate can be greater than 65 micrometers per year. In-vivo corrosion rates can be estimated by placing the stent in Ringer's solution (25 L of water containing NaCl (710 g), MgSO4 (205 g), MgCl2_{—}6H2O (107.5 g), CaCl2_{—}6H2O (50 g)) according to the standard protocol ASTM-D1141-98. Corrosion rates can also be measured by standard electyochemical methods, potentiodynamic, and impedance. For example, In vitro and in vivo corrosion measurements of magnesium alloys, Frank Witte, Jens Fischer, Jens Nellesen, Horst-Artur Crostack, Volker Kease, Alexander Pisch, Felix Beckmann, and Henning Windhagen, Biomaterials 27 (2006) pp. 1013-18 describes methods for corrosion measurements, which is hereby incorporated by reference.

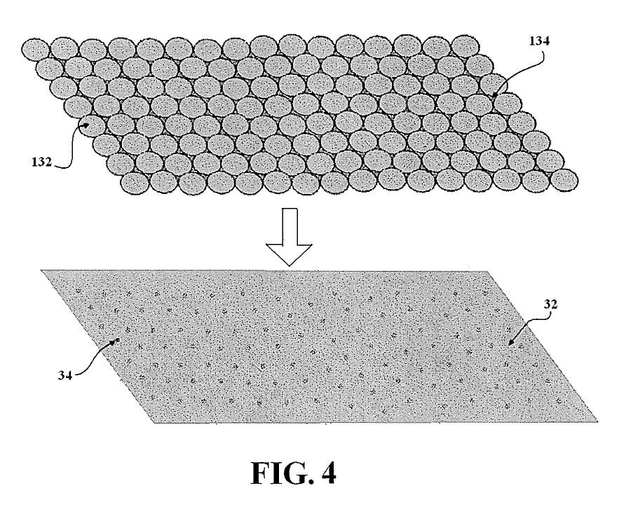

A stent having inter-granular deposits 34 of a salt or clay within a matrix 32 of a bioerodible metal can be produced by a sintering process. For example, many common sintering processes use binders to shape parts prior to sintering. These typical binders include ingredients that are gassed out during the sintering process, which usually includes temperatures of between 1200° Celsius and 1300° Celsius. A structure including a matrix of a bioerodible metal 32 including inter-granular deposits 34 of a salt or a clay can be made by including a salt or clay in a binder. The binder 134 is mixed within a metallic powder including bioerodible metal particles 132, so that the binder 134 is positioned in the void spaces between adjacent particles, as shown in FIG. 4. After the sintering process, a salt or clay within the binder can remain as salt or clay inclusions or precipitations, as shown in FIG. 4. In a sintering process, the included salt or clay should be selected so that it does not gas out in that particular sintering process. For example, sodium fluoride, sodium chloride, iron chloride, iron fluoride, and potassium sulfate can remain after sintering processes. For example, an iron matrix 32 including sodium chloride inclusions 34 can be produced by including sodium chloride in a binder 134 used to shape iron powder particles 132. In some embodiments, the iron powder can be carbonyl iron powder, which is available from BASF.

The process conditions used during the sintering process can impact the size, spacing, and arrangement of the resulting deposits 34 within the bioerodible metal 32. For example, higher sintering temperatures can result in a greater amount of diffusion of the salt or clay within the matrix 32. Additionally, the bioerodible metal particle size distribution can impact the size and spacing of the deposits 34. As shown in FIG. 4, the binder 134, when mixed with bioerodible metal particles, is positioned in the void space between adjacent particles. Larger particle sizes result in larger void spaces, which can result in larger deposit sizes. For example, carbonyl iron powder is available from BASF in multiple particle size distributions. This can result in an average deposit size of between 5 nanometers and 200 nanometers. In some embodiments, the average deposit size is between 50 and 150 nanometers (e.g., about 100 nanometers). Other methods of positioning salt or clay deposits within a bioerodible metal matrix are also possible, which can result in salt or clay deposits of different dimensions.

The stent can also be produced using micro-metal injection molding (“μ-MIM”) or micro-metal extrusion (“μ-ME”). A description of μ-MIM can be found in Influence of Multistep Theremal Control in Metal Powder Injection Moulding Process, L. W. Houmg, C. S. Wang, and G. W. Fan, Powder Metallurgy, 2008, Vol. 51, No. 1, pp. 84-88 and Development of Metal/Polymer Mixtures for Micro Powder Injection Moulding, C. Quinard, T. Barriere, and J. C. Gelin, CP907, 10^{th} ESAFORM Conference on Material Forming, edited by E. Cueto and F. Chinesta, pp. 933-38, which are hereby incorporated by reference. The μ-ME process is similar, but involves the extrusion, rather than injection molding, of the constituents. A stent accordingly to the instant disclosure can be formed by using μ-MIM or μ-ME to form a tube including a bioerodible metal matrix and a salt or clay within the bioerodible metal matrix. For example, the feedstock for the μ-MIM or μ-ME process can include 39-55 volume percent binder, 44-60 volume percent iron, and between 1 and 10 volume percent milled sodium chloride having an average particle size of less than 100 micrometers (e.g., between 5 and 75 micrometers). Carbonyl iron having a 1 micrometer diameter from BASF in the HQ grade is, for example, suitable for use as the iron in the feedstock for the μ-MIM or μ-ME processes. The feedstock can be premixed and kneaded prior to forming the tube using the μ-MIM or μ-ME processes. When using a μ-ME process, shear roll extrusion can be used for a final homogenization and granulation before the extrusion of a tube. The formed tube can then be cut to form struts and polished to form a finished stent.

For example, particles of Iron alloyed with about three percent Silicon (Fe-3Si) can be mixed with particles that include a binder (e.g., polymer and wax) and about two percent potassium sulfate (K₂SO₄). The particles of the Iron-Silicon alloy can have a diameter of between about 4 μm and about 20 μm while the particles of the potassium sulfate and binder can have a diameter of less than 4 μm (e.g., about 1 to 2 μm in diameter). The mixture of particles can be extruded to near net-shape using metal injection molding (“MIM”), μ-MIM, and/or μ-ME. The binder can be removed with hexane. The shaped material (e.g., the rod or tube produced by MIM) can be sintered between 1050° C. and 1200° C. The sintered material can then be further processed into tubes having the desired dimensions by drawing. This process can produce a density of greater than 97 percent.

The distribution of the deposits 34 can also be varied within the bioerodible metal matrix 32. For example, FIG. 3B depicts embodiments of a stent strut having bioerodible metal portions 33 that do not include deposits. The deposits within a bioerodible metal portion can be patterned to result in a desired erosion pattern. For example, different binders having different amounts and/or different types of salts or clays can be used in different portions of the bioerodible metal powder. The use of different binders having different amounts or types of salts or clays can allow for the design of a bioerodible portion having a desired erosion pattern. The μ-MIM process can be used to create a mixture of bioerodible metal powder and binder having different binders in different portions of the mixture. The distributions of deposits within a bioerodible metal matrix can allow for a bioerodible portion to have a desired erosion pattern when implanted within a physiological environment.

The bioerodible metal matrix 32 can also include more than one bioerodible metal. In some embodiments, a secondary bioerodible metal can form a gradient within the bioerodible portion from a first bioerodible metal to a second bioerodible metal. In some embodiments, multiple bioerodible metals can form a galvanic couple, which can further impact the corrosion characteristics of the bioerodible portion. In other embodiments, the bioerodible metal can be an alloy including a gradient in the concentration of the constituents of the alloy. For example, additional metallic elements can be further embedded within the bioerodible metal matrix by a plasma sintering process whereby the sintered body is heated by bombardment of ions out of the plasma and the plasma includes metallic atoms derived by a sputtering process from a secondary cathode. In some embodiments, these additional metallic elements can form a galvanic couple within the bioerodible metal. The plasma sintering process can create a bioerodible metal alloy matrix having a gradient in the amount of the additional metallic elements normal to the surface of the sintered device.

FIG. 3C depicts another embodiment of a stent strut cross-section having polymer deposits 35 within a bioerodible metal matrix 32. The polymer deposits 35 include a polymer having acidic functional groups that produce a localized acidic environment when exposed to a body fluid. Once a polymer deposit 35 becomes exposed to body fluid during the erosion process of the bioerodible metal, the polymer deposit can swell with body fluid and create a localized acidic environment, which can accelerate the erosion rate of the bioerodible metal. The acceleration of the erosion rate of the bioerodible metal along the interface between the polymer deposits 35 and the bioerodible metal matrix 32 results in the erosion of the bioerodible portion from within.

FIG. 3D depicts another embodiment of a stent strut cross-section having polymer/electrolyte deposits 36 within a bioerodible metal matrix 32. The polymer/electrolyte deposits 36 include a polymer that forms a galvanic couple with the bioerodible metal of the bioerodible metal matrix 32 once a body fluid from within the physiological environment contacts the polymer/electrolyte deposit 36. Within the galvanic couple, the polymer acts as the cathode and the bioerodible metal acts as the anode, which results in the preferential erosion of the bioerodible metal along the interface of the polymer/electrolyte deposits 36 once a body fluid has penetrated into the polymer/electrolyte deposit. The polymer/electrolyte deposits 36 can also include a salt 34 that ionizes when exposed to a body fluid to make the polymer conductive. The ionization of the salt further provides electrolytes within each deposit 36 to increase the efficiency of the oxidation/reduction reaction between the polymer and the bioerodible metal. For example, polymer/electrolyte deposits 36 can include PEO loaded with chloride based salts, such as magnesium chloride or iron chloride.

The preferential erosion of the stent along the interface of internal deposits within a bioerodible metal matrix 32 allows the stent to erode from the inside, resulting in an increased erosion rate after an initial slower erosion rate. Initially upon implantation, internal deposits 35 or 36 remain separated from body fluids within the physiological environment, thus preventing any oxidation/reduction reaction between the polymer and the bioerodible metal. As the outer surface of the bioerodible metal matrix 32 erodes, however, crevices and cracks form and eventually allow for the diffusion of body fluids into the deposits. The polymers can, in some embodiments, swell with the body fluid and allow for internal erosion of the bioerodible metal in addition to the external erosion of the bioerodible metal, with a faster internal erosion rate. By having a stent with a first erosion rate that is slower than a second erosion rate, the stent strut can be designed to have smaller initial dimensions than a stent having a constant erosion rate because the first erosion rate preserves the structural properties of the stent during an initial healing process during the initial erosion period. The accelerated erosion period after body fluid has entered the deposits then reduces the amount of time that a weakened stent strut remains present within a body passageway.

Localized acidic regions can also be positioned along the outer surface of a bioerodible metal to increase the erosion rate at particular locations in the bioerodible portion. For example, FIG. 3E depicts an embodiment of a stent strut cross-section including a bioerodible metal portion 31 having surface deposits 37. As shown, the surface deposits are deposited within pits in the surface of the bioerodible metal. The surface deposit can include a polymer, a salt, a clay, or a combination thereof. Furthermore, FIG. 3F depicts an embodiment of a stent having an outer surface polymer coating 38 that includes localized acidic group clusters 39. The localized acidic group clusters 39 have a higher percentage of acidic functional groups then the remainder of the polymer, which can allow for a preferential erosion of the bioerodible metal along the interface between the localized acidic group clusters 39 and the bioerodible metal portion 31. The location of the surface deposits 37 and localized acidic group clusters 39 can impact overall erosion characteristics of the bioerodible portion. In some embodiments, surface deposits 37 or localized acidic group clusters 39 can be positioned to direct an erosion path towards an internal deposit 35 or 36.

The stent body, in some embodiments, can have different portions of different struts having different erosion rates so that the stent can degrade in a controlled manner. For example, certain portions of different stent struts can include deposits of salt, clay, and/or polymer within a matrix of the bioerodible metal, or a higher percentage thereof. As shown in FIG. 5A, the connectors 24 of the stent 20 can include corrosion enhancing regions 26 having a higher percentage of salt, clay, or polymer. Such an arrangement can allow for the connectors 24 to degrade first, which can increase the flexibility of the stent along the longitudinal axis while radial opposition to the vessel wall is maintained. FIG. 5B depicts the stent after the erosion of the connectors 24, leaving the unconnected bands 22 that can still provide radial vessel opposition.

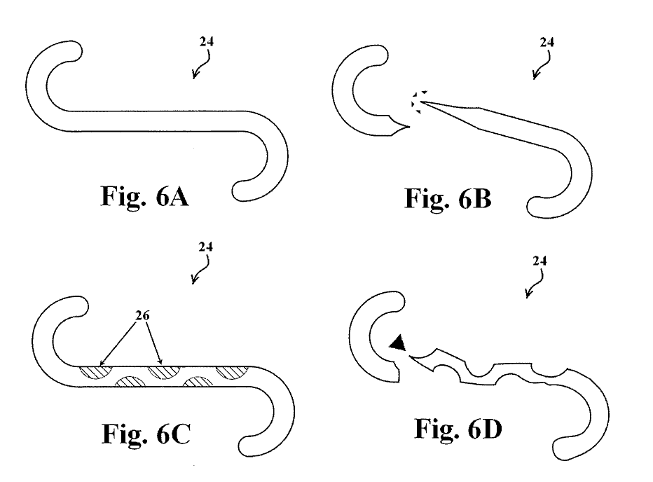

FIGS. 6A-6D show the difference between the uncontrolled erosion of a stent strut (e.g., portions of a band or a connector) and the erosion of a stent strut having corrosion enhancing regions 26. As shown by FIGS. 6A and 6B, uncontrolled degradation can cause struts to narrow and break in localized areas producing a sharp strut that retains its columnar strength, which can produce a piercing risk. A stent strut having spaced corrosion enhancing regions 26, however, can reduce the piercing risk. As shown in FIGS. 6C and 6D, corrosion enhancing regions 26 can erode to produce a strut having low columnar strength. Because the corrosion enhancing regions 26 can erode to produce an easily bent strut, the erosion of the stent strut produces a lower piercing risk.

In some embodiments, the stent 20 can also include a therapeutic agent. In some embodiments, the therapeutic agent can be incorporated into the bioerodible portion. For example, the therapeutic agent can be incorporated in the bioerodible polymer and elude as the bioerodible polymer degrades under physiological conditions, for example within deposits 35, 36, or 37, or within coating 38.

The stent 20 can, in some embodiments, be adapted to release one or more therapeutic agents. The term “therapeutic agent” includes one or more “therapeutic agents” or “drugs.” The terms “therapeutic agents” and “drugs” are used interchangeably and include pharmaceutically active compounds, nucleic acids with and without carrier vectors such as lipids, compacting agents (such as histones), viruses (such as adenovirus, adeno-associated virus, retrovirus, lentivirus and a-virus), polymers, antibiotics, hyaluronic acid, gene therapies, proteins, cells, stem cells and the like, or combinations thereof, with or without targeting sequences. The delivery mediated is formulated as needed to maintain cell function and viability. A common example of a therapeutic agent includes Paclitaxel.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721).

In use, a stent can be used, e.g., delivered and expanded, using a catheter delivery system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Sentinol® system, available from Boston Scientific Scimed, Maple Grove, Minn.

All publications, references, applications, and patents referred to herein are incorporated by reference in their entirety.

A number of embodiments have been described. Nevertheless, it will be understood that various modifications may be made without departing from the spirit and scope of this disclosure. Accordingly, other embodiments are within the scope of the following claims.

What is claimed is:

1. A medical implant comprising a bioerodible portion adapted to degrade under physiological conditions, the bioerodible portion comprising:
a bioerodible metal matrix; and
a salt or clay within the bioerodible metal matrix.

2. The medical implant of claim 1, wherein the bioerodible portion comprises a chloride salt, a fluoride salt, a sulfate, or a combination thereof.

3. The medical implant of claim 1, wherein the bioerodible portion comprises a salt having a melting point of greater than 700 degrees Celsius.

4. The medical implant of claim 1, wherein the bioerodible portion comprises a salt selected from the group consisting of iron chloride, magnesium chloride, sodium chloride, iron fluoride, sodium fluoride, sodium bicarbonate, sodium sulfate, potassium sulfate, calcium phosphate, magnesium acetate, magnesium citrate, lidocanine hydrochloride, dexamethasone sodium phosphate, paclitaxel mesylate and combinations thereof.

5. The medical implant of claim 1, wherein the bioerodible portion comprises a clay selected from the group consisting of calcium permanaganates.

6. The medical implant of claim 1, wherein the bioerodible portion is essentially free of polymer.

7. The medical implant of claim 1, wherein the bioerodible portion comprises a polymer matrix within the bioerodible metal matrix, the salt or clay being within the polymer matrix.

8. The medical implant of claim 7, wherein the polymer matrix comprises a polymer selected from the group consisting of poly(ethylene oxide), polylactic acid, poly(lactic-co-glycolic acid), polycaprolactone, polycaprolactone-polylactide copolymer, polycaprolactone-polyglycolide copolymer, polycaprolactone-polylactide-polyglycolide copolymer, polylactide, polycaprolactone-poly(β-hydroxybutyric acid) copolymer, poly(β-hydroxybutyric acid) and combinations thereof.

9. The medical implant of claim 1, wherein the bioerodible metal comprises iron or an alloy thereof.

10. The medical implant of claim 9, wherein the bioerodible portion has an erosion rate of greater than thirty micrometers per year when submerged in Ringer's solution at ambient temperature.

11. The medical implant of claim 1, wherein the medical implant consists essentially of the bioerodible portion.

12. The medical implant of claim 1, wherein the medical implant is a stent.

13. A medical implant comprising a bioerodible portion adapted to degrade under physiological conditions, the bioerodible portion comprising:
a bioerodible metal that degrades under physiological conditions; and
an agent that creates a localized acidic environment when exposed to a body fluid under physiological conditions, the localized acidic environment accelerating the erosion of the bioerodible metal in the vicinity of the localized acidic environment, the agent selected from the group consisting of salts, clays, polymers, an combinations thereof.

14. The medical implant of claim 13, wherein the agent is a salt selected from the group consisting of iron chloride, magnesium chloride, sodium chloride, iron fluoride, sodium fluoride, sodium bicarbonate, sodium sulfate, calcium phosphate, magnesium acetate, magnesium citrate, lidocanine hydrochloride, dexamethasone sodium phosphate, paclitaxel mesylate and combinations thereof.

15. The medical implant of claim 13, wherein the agent is a polymer selected from the group consisting of poly(ethylene oxide), polylactic acid, poly(lactic-co-glycolic acid), polycaprolactone, polycaprolactone-polylactide copolymer, polycaprolactone-polyglycolide copolymer, polycaprolactone-polylactide-polyglycolide copolymer, polylactide, polycaprolactone-poly(β-hydroxybutyric acid) copolymer, poly(β-hydroxybutyric acid) and combinations thereof.

16. The medical implant of claim 13, wherein the bioerodible metal comprises iron or an alloy thereof.

17. The medical implant of claim 13, wherein the agent is within a matrix of the bioerodible metal.

18. The medical implant of claim 13, wherein the agent is deposited on an outer surface of the bioerodible metal.

19. The medical implant of claim 18, wherein the outer surface comprises surface pits and the agent is deposited within the surface pits.

20. The medical implant of claim 13, wherein the medical implant is a stent.
